(19) United States
(12) Patent Application Publication
(10) Pub. No.: US 2022/0313209 A1
(43) Pub. Date: Oct. 6, 2022

(54) SYSTEM FOR MEDICAL DIAGNOSIS USING ARTIFICIAL INTELLIGENCE AND DEEP LEARNING APPROACH

(71) Applicants: Mahmoud Elsayed Ragab, Jeddah (SA); Hani M. Zubair Choudhry, Jeddah (SA); Amer Hamzah Asseri, Jeddah (SA); Maha Farouk Sabir, Jeddah (SA); Abdullah S. AL-Malaise AL-Ghamdi, Jeddah (SA); Romany F. Mansour, El-Kharga (EG); Amit Kumar Tyagi, Chennai (IN)

(72) Inventors: Mahmoud Elsayed Ragab, Jeddah (SA); Hani M. Zubair Choudhry, Jeddah (SA); Amer Hamzah Asseri, Jeddah (SA); Maha Farouk Sabir, Jeddah (SA); Abdullah S. AL-Malaise AL-Ghamdi, Jeddah (SA); Romany F. Mansour, El-Kharga (EG); Amit Kumar Tyagi, Chennai (IN)

(21) Appl. No.: 17/806,573

(22) Filed: Jun. 13, 2022

(51) Int. Cl. A61B 8/12; A61B 6/00; A61B 5/00

(52) U.S. Cl. CPC A61B 8/12; A61B 6/504; A61B 5/0084; A61B 5/02007

(57) ABSTRACT

The medical diagnostic system comprises a deep neural network model trained with catheter hardness data, lesion hardness data, and operation time to complete catheter treatment; an irradiation energy emitting device to calculate irradiation energy of a patient; a control unit to identify a plurality of lesions from irradiation energy data; and a catheter to insert into an artery in the patient's arm, wherein the catheter tip is positioned to at least the patient's aortailiac bifurcation, wherein a therapeutic catheter is introduced into a catheter lumen and the therapeutic catheter tip is projected from the catheter tip thereby the harder lesion is initially treated, and the therapeutic catheter tip of the therapeutic catheter is projected from the catheter tip to treat the softer lesion.

### TECHNICAL FIELD

The present disclosure relates to a system for medical diagnosis using artificial intelligence and deep learning approach.

### BACKGROUND

In order for a surgeon to access a patient's heart, traditional procedures for cardiac device implantation necessitate cutting a somewhat wide aperture in the sternum or thoracic cavity. These operations often need cardiac arrest and cardiopulmonary bypass (the use of a heart-lung bypass machine to oxygenate and circulate the patient's blood). In most cases, these treatments are followed by a lengthy hospital stay and healing period for the patient. Furthermore, tissue adherences from the initial operation may enhance the risks of subsequent valve replacement surgeries, such as stroke and mortality, by increasing the hazards of recovery.

For heart valve replacement, both synthetic and biological valves have been employed. Synthetic valves seldom fail, although they do need lifelong anticoagulant therapy to prevent blood from clotting in and around the replacement valve. Anticoagulant therapy limits patients' activities and can lead to a variety of additional problems. Other known technologies sometimes necessitate complicated implant procedures and are extremely time sensitive, since they may require temporary artificial heart pumping help. To enable for the insertion of a medical device for the heart, it is normal procedure to induce cardiac arrest. The patient will be kept alive by external equipment during this treatment.

In the view of the forgoing discussion, it is clearly portrayed that there is a need to have a system for medical diagnosis using artificial intelligence and deep learning approach.

### BRIEF SUMMARY

The present disclosure seeks to provide a medical diagnostic system for treating the blood vessel by an intervention procedure using artificial intelligence and deep learning approach.

In an embodiment, a medical diagnostic system is disclosed. The system includes a deep neural network model trained with catheter hardness data, lesion hardness data, and operation time to complete catheter treatment. The system further includes an irradiation energy emitting device to calculate irradiation energy of a patient. The system further includes a control unit to identify a plurality of lesions from irradiation energy data, the plurality of lesions including one or more lesions in each of the plurality of bifurcated lumens, obtain lesion hardness data on each of the plurality of lesions from irradiation energy data, and determine a lesion to be treated first among the plurality of lesions based on the lesion hardness data of each of the one or more lesions in each of the plurality of bifurcated lumens, wherein based on the lesion hardness data of each of the one or more lesions in each of the plurality of bifurcated lumens, select a lesion to be treated later among the plurality of lesions, the lesion to be treated later being in another of the plurality of bifurcated lumens. The system further includes a catheter to insert into an artery in the patient's arm, wherein the catheter tip is positioned to at least the patient's aortailiac bifurcation, wherein a therapeutic catheter is introduced into a catheter lumen and the therapeutic catheter tip is projected from the catheter tip thereby the harder lesion is initially treated, and the therapeutic catheter tip of the therapeutic catheter is projected from the catheter tip to treat the softer lesion.

In another embodiment, the hardness of the catheter tip is selected based on the hardness of each of the one or more lesions in each of the plurality of bifurcation lumens.

In another embodiment, the irradiation energy collected via the patient is detected upon irradiating it with irradiation energy and collecting irradiation energy data on the patient-based on a changing irradiation energy.

In another embodiment, the lesion to be treated first is selected from a group of lesions based on the stenosis rate data, wherein the stenosis rate data is acquired on the plurality of lesions using the deep neural network model.

In another embodiment, a first therapeutic catheter is used to treat softer lesion is and a second therapeutic catheter is used to treat harder lesion.

In another embodiment, the selected bifurcation is an aortoiliac bifurcation when the primary lumen is an aorta, the plurality of bifurcated lumens are right and left lower limb arteries, and the right and left lower limb arteries each have lesions.

In another embodiment, the irradiation energy is selected from a group of X-rays, ultrasonic waves, infrared rays, visible light, magnetic field lines, and the like, wherein the X-ray is preferable if the irradiation energy is far away from the human body, whereas ultrasonic waves and visible light are better if the irradiation energy is in touch with or within the human body, wherein a combination of ultrasonic waves and near-infrared rays is employed when one or more energies are used.

An object of the present disclosure is to treat the blood vessel by an intervention procedure.

Another object of the present disclosure is to diagnose which of one or more lesions in each of a plurality of blood vessels bifurcated from a blood vessel having bifurcations is to be treated first for treating the blood vessel by an intervention procedure.

Another object of the present disclosure is to optimally operating a centrifugal implanted blood pump from left atrium to aorta.

Yet another object of the present disclosure is to deliver an expeditious and cost-effective medical diagnostic system.

To further clarify advantages and features of the present disclosure, a more particular description of the disclosure will be rendered by reference to specific embodiments thereof, which is illustrated in the appended drawings. It is appreciated that these drawings depict only typical embodiments of the disclosure and are therefore not to be considered limiting of its scope. The disclosure will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a block diagram of a system for medical diagnosis using artificial intelligence and deep learning approach in accordance with an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this disclosure belongs. The system, methods, and examples provided herein are illustrative only and not intended to be limiting.

Embodiments of the present disclosure will be described below in detail with reference to the accompanying drawings.

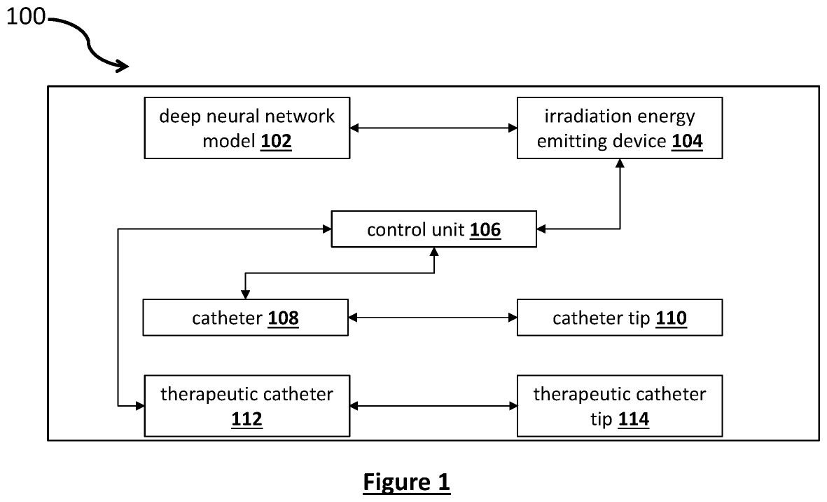

Referring to FIG. 1, a block diagram of a system for medical diagnosis using artificial intelligence and deep learning approach is illustrated in accordance with an embodiment of the present disclosure. The system 100 includes a deep neural network model 102 trained with catheter hardness data, lesion hardness data, and operation time to complete catheter treatment.

In an embodiment, an irradiation energy emitting device 104 is connected to the deep neural network model 102 to calculate irradiation energy of a patient.

In an embodiment, a control unit 106 is connected to the irradiation energy emitting device 104 to identify a plurality of lesions from irradiation energy data, the plurality of lesions including one or more lesions in each of the plurality of bifurcated lumens, obtain lesion hardness data on each of the plurality of lesions from irradiation energy data, and determine a lesion to be treated first among the plurality of lesions based on the lesion hardness data of each of the one or more lesions in each of the plurality of bifurcated lumens, wherein based on the lesion hardness data of each of the one or more lesions in each of the plurality of bifurcated lumens, select a lesion to be treated later among the plurality of lesions, the lesion to be treated later being in another of the plurality of bifurcated lumens.

In an embodiment, a catheter 108 is connected to the control unit 106 to insert into an artery in the patient's arm, wherein the catheter tip is positioned to at least the patient's aortailiac bifurcation, wherein a therapeutic catheter 112 is introduced into a catheter lumen and the therapeutic catheter tip 114 is projected from the catheter tip 110 thereby the harder lesion is initially treated, and the therapeutic catheter tip 114 of the therapeutic catheter 112 is projected from the catheter tip 110 to treat the softer lesion.

In another embodiment, the hardness of the catheter tip 110 is selected based on the hardness of each of the one or more lesions in each of the plurality of bifurcation lumens.

In another embodiment, the irradiation energy collected via the patient is detected upon irradiating it with irradiation energy and collecting irradiation energy data on the patient-based on a changing irradiation energy.

In another embodiment, the lesion to be treated first is selected from a group of lesions based on the stenosis rate data, wherein the stenosis rate data is acquired on the plurality of lesions using the deep neural network model 102.

In another embodiment, a first therapeutic catheter is used to treat softer lesion is and a second therapeutic catheter is used to treat harder lesion.

In another embodiment, the selected bifurcation is an aortoiliac bifurcation when the primary lumen is an aorta, the plurality of bifurcated lumens are right and left lower limb arteries, and the right and left lower limb arteries each have lesions.

In another embodiment, the irradiation energy is selected from a group of X-rays, ultrasonic waves, infrared rays, visible light, magnetic field lines, and the like, wherein the X-ray is preferable if the irradiation energy is far away from the human body, whereas ultrasonic waves and visible light are better if the irradiation energy is in touch with or within the human body, wherein a combination of ultrasonic waves and near-infrared rays is employed when one or more energies are used.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A system for medical diagnosis using artificial intelligence and deep learning approach, said system comprises:
a deep neural network model trained with catheter hardness data, lesion hardness data, and operation time to complete catheter treatment;
an irradiation energy emitting device to calculate irradiation energy of a patient;
a control unit to identify a plurality of lesions from irradiation energy data, said plurality of lesions including one or more lesions in each of said plurality of bifurcated lumens, obtain lesion hardness data on each of said plurality of lesions from irradiation energy data, and determine a lesion to be treated first among said plurality of lesions based on said lesion hardness data of each of said one or more lesions in each of said plurality of bifurcated lumens, wherein based on said lesion hardness data of each of said one or more lesions in each of said plurality of bifurcated lumens, select a lesion to be treated later among said plurality of lesions, said lesion to be treated later being in another of said plurality of bifurcated lumens; and
a catheter to insert into an artery in said patient's arm, wherein said catheter tip is positioned to at least said patient's aortailiac bifurcation, wherein a therapeutic catheter is introduced into a catheter lumen and said therapeutic catheter tip is projected from said catheter tip thereby said harder lesion is initially treated, and said therapeutic catheter tip of said therapeutic catheter is projected from said catheter tip to treat said softer lesion.

2. The system as claimed in claim 1, wherein said hardness of said catheter tip is selected based on said hardness of each of said one or more lesions in each of said plurality of bifurcation lumens.

3. The system as claimed in claim 1, wherein said irradiation energy collected via said patient is detected upon irradiating it with irradiation energy and collecting irradiation energy data on said patient-based on a changing irradiation energy.

4. The system as claimed in claim 1, wherein the lesion to be treated first is selected from a group of lesions based on said stenosis rate data, wherein said stenosis rate data is acquired on said plurality of lesions using said deep neural network model.

5. The system as claimed in claim 1, wherein a first therapeutic catheter is used to treat softer lesion is and a second therapeutic catheter is used to treat harder lesion.

6. The system as claimed in claim 1, wherein said selected bifurcation is an aortoiliac bifurcation when said primary lumen is an aorta, said plurality of bifurcated lumens are right and left lower limb arteries, and said right and left lower limb arteries each have lesions.

7. The system as claimed in claim 1, wherein said irradiation energy is selected from a group of X-rays, ultrasonic waves, infrared rays, visible light, magnetic field lines, and the like, wherein said X-ray is preferable if said irradiation energy is far away from said human body, whereas ultrasonic waves and visible light are better if said irradiation energy is in touch with or within said human body, wherein a combination of ultrasonic waves and near-infrared rays is employed when one or more energies are used.
